(19) United States
(12) Patent Application Publication
(10) Pub. No.: US 2005/0124901 A1
(43) Pub. Date: Jun. 9, 2005

(54) Method and apparatus for electrophysiological and hemodynamic real-time assessment of cardiovascular fitness of a user

(76) Inventors: Dale Julian Misczynski, Austin, TX (US); Vladislav Bukhman, East Northport, NY (US)

Correspondence Address: Dale J. Misczynski, 1800 Barton Creek Blvd, Austin, TX, 78735-1606, US

(21) Appl. No.: 10/990,847

(22) Filed: Nov. 17, 2004

Related U.S. Application Data
(60) Provisional application No. 60/527,179, filed on Dec. 5, 2003.

(51) Int. Cl. A61B 5/04; A61B 5/02

(52) U.S. Cl. 600/509; 600/526; 600/390

(57) ABSTRACT

The invention relates to a method and apparatus for real-time assessment of one or more electrical and one or more hemodynamic parameters for the evaluation of cardiovascular fitness. The invention comprises simultaneous analysis of electrical (ECG) and hemodynamic (Impedance Cardiogram—ICG) activities of the heart by evaluating said activities using characteristic points detection method. These points are used for the calculation of electrophysiological parameters such as heart rate (HR) and heart rate variability (HRV) and obtaining the data needed for calculation of hemodynamic parameters such as stroke volume (SV) and cardiac output (CO). The characteristic points detection method significantly improves tolerance to noise and artifacts associated with body movements, thereby enabling the user to assess his/her cardiovascular fitness while being evaluated.

### CROSS REFERENCE TO RELATED APPLICATIONS

This patent application is based upon Provisional Patent Application Ser. No. 60/527,179 filed on Dec. 05, 2003

### BACKGROUND OF THE INVENTION

This invention relates to the field of heart rate monitors, and in particular to heart rate monitors used for electrophysiological and hemodynamic assessment of the cardiovascular fitness of athletes, sport enthusiasts and health conscience people.

Cardiovascular fitness refers to the quantity of work that can be performed by the muscles, which is critically dependent on the volume of blood that can be delivered by the heart.

A large number of products are available commercially from manufacturers like Polar, Timex, Acumen, Cardiosport, Performance, Sensor Dynamics, Sports Instruments and Vetta, such as the S810i by Polar, for the evaluation of cardiovascular fitness based on the user's heart rate. These devices, which are called heart rate monitors (HRM), detect the electrical activity of the heart through electrocardiogram type electrodes mounted in a chest strap. The filtered and amplified electrical signals are then transmitted to a wristwatch-type device or a device mounted on exercise equipment for further processing and display of calculated heart rate and other relevant information.

Use of heart rate monitors for the evaluation of cardiovascular fitness is based on the assumption that the volume of blood, and hence the volume of oxygen delivered to the muscles is directly proportional to the heart rate. For example, a higher heart rate results in more blood being supplied to the muscles. Unfortunately, this assumption is only correct for the ideal situation and it's not true in many occasions such as endurance training, maximum workloads, cardiovascular abnormalities, extreme or unusual environments, effect of caffeine, and people taking medications.

While it is useful to know your heart rate when exercising, it is not the only parameter that should be monitored for assessment of cardiovascular fitness. Heart rate does not necessarily give an accurate picture of cardiovascular fitness since the main factor in such a determination, how much blood the heart is actually able to supply to your body per minute (cardiac output), is not taken into account. Cardiac Output is calculated using the following equation:
CO=SV*HR (1)
Where:
CO—Cardiac Output (liter/min)
SV—Stroke Volume, the volume of blood ejected from the heart due to contraction of the left ventricular,
HR—Heart Rate, number of heart beats per minute.

Obtaining the actual stroke volume in a clinical setting is complicated. Consequently, cardiac output is traditionally derived in a clinical setting use any of the following four methods.

Fick Method

Cardiac Output=[oxygen absorbed by the lungs (ml/min)]/[arteriovenous oxygen difference (ml/liter of blood)].

Oxygen consumption is derived by measuring the expired gas volume over a known period of time. The arteriovenous oxygen difference is obtained by taking blood samples or by examining the oxygen context of the user's expired and inspired gas. These methods are difficult to implement. For example, unless the patient has an endotracheal tube, measurements may be faulty because of leakage around the facemask or mouthpiece. Those of ordinary skill in the art will fully appreciate cardiac output measurement by the Fick method. Further details may be found in The Textbook of Medical Physiology, 7^{th} Ed., p. 284 by Arthur C. Guyton, which is hereby incorporated by reference.

Thermodilution Method

This technique for measuring the cardiac output requires the monitoring of temperature changes after bolus injection of a cold liquid into the blood stream. The injection is made via a catheter that contains a thermistor mounted at its tip. The thermistor measures the sequential changes in temperature which are then plotted over time. The cardiac output is inversely related to the area under the thermodilution curve. This is the standard method for monitoring cardiac output in an intensive care unit. Those of ordinary skill in the art will fully appreciate this method. Similar methods include the Indicator Dilution Method as described in The Textbook of Medical Physiology, 7^{th} Ed., p. 284-285 by Arthur C. Guyton, which is hereby incorporated by reference.

Echocardiography

This method can be used to derive cardiac output from the measurement of blood flow velocity by recording the Doppler shift of ultrasound reflected from blood cells as they pass through a vessel. The time/flow integral, which is the integral of instantaneous blood flow velocities during one cardiac cycle, is obtained for the blood flow in the left ventricular outflow tract (other sites can be used). This is multiplied by the cross-sectional area of the tract and the heart rate to give cardiac output. The main disadvantages of this method are that a skilled operator is needed, the probe is large and therefore heavy sedation or anesthesia is needed, the equipment is very expensive and the probe cannot be fixed so as to give continuous cardiac output readings without an expert user being present. Those of ordinary skill in the art will fully appreciate this method. The Textbook of Medical Physiology, 7^{th} Ed., p. 284 by Arthur C. Guyton, which is hereby incorporated by reference.

Thoracic Bioimpedance Technology

This method has the advantages of providing continuous cardiac output measurement at limited risk to the patient. A small, high frequency current is passed through the thorax via electrodes placed on the skin. Contraction of the heart produces a cyclical change in thoracic impedance. Sensing electrodes are used to measure the changes in impedance within the thorax. A constant current generator establishes a fixed level for I(o). The resulting voltage change, V(t), is used to calculate impedance. Because the impedance is assumed to be purely resistive, the total impedance, Z, is calculated by Ohm's Law. The normal impedance value for an adult is 20-48 ohms with a current frequency of 50-100 Hz. The total impedance is derived from a constant base impedance, Zₒ, and time-varying impedance, Z(t), as shown in equation (2) below and FIG. 1:

$$Z = \frac{V ( t )}{I ( o )} = {Z}_{o} + Z ( t ) \qquad ( 2 )$$

Zₒ 140 reflects constant resistivity of tissue and bones. Time-varying impedance Z(t) 130 reflects changes in resistivity of portions of the arterial system as blood flows through the aorta.

The aforementioned impedance values may then be used to calculate stroke volume using the Kubicek equation (3) or any of its modifications like, for example, the Gundarov equation (4):

$$\begin{matrix}SV = ρ ( \frac{{L}^{2}}{{Z}_{o}} ) LVET \frac{ⅆ Z ( t )}{ⅆ {t}_{max}} & ( 3 ) \\ SV = 0.9 ρ κ \frac{{Q}^{2} L}{{Z}_{o}^{2}} LVET \frac{ⅆ Z ( t )}{ⅆ {t}_{max}} & ( 4 )\end{matrix}$$

Where:
L=distance between sensing electrodes (cm),
LVET=left ventricular ejection time,
Zₒ=base impedance in (ohms),

$$\begin{matrix}\frac{ⅆ Z ( t )}{ⅆ {t}_{max}} = magnitude of the largest negative derivative of \\ the impedance change ,\end{matrix}$$
Z(t) occurring during systole (ohms/s)
ρ=resistivity of blood (ohms/cm),
δ=weight correction factor,
H=height of the user,
κ=torso correction factor
Q=circumference of torso.

LVET is the time between the opening and closing of the aortic valve. These times can be identified using two characteristic points of the ICG. Point B corresponds with the aortic valve opening. Point X corresponds with the aortic valve closing (FIG. 4).

$$\frac{ⅆ Z ( t )}{ⅆ {t}_{max}}$$

corresponds to characteristic point C of the ICG (FIG. 4), which is point of maximum amplitude in the cardiac cycle.

As indicated in equation 1, once stroke volume is known, it may be multiplied by the heart rate to determine cardiac output and consequently, cardiovascular fitness.

In contrast to the Fick method and the other cardiac output methods previously described, only the thoracic bioimpedance technology, also called impedance cardiography, is practically useful for real-time assessment of cardiovascular fitness during workload. Still, there are drawbacks associated with this technology:
Unfortunately, the cyclical change in impedance cardiography is about 0.5% to 1%. This yields a low signal to noise ratio. Consequently, the impedance signal is vulnerable to noise and artifacts.
The user should lie still in order to limit noise and artifacts associated with muscle contractions.
The impedance cardiography procedure requires positioning of at least 4 pairs of electrodes in the neck and thoracic areas. These electrodes must then be connected to a signal processing unit, thereby restricting the user's freedom of movement.
The calculation of stroke volume, and consequently cardiac output, using impedance cardiography requires ECG derived data such as RR interval and Q wave onset for detection of left ventricular ejection time and maximum impedance. ECG-based technologies incorporated in current heart rate monitors only detect electrical spikes associated with the heartbeat and not values such as RR interval and Q wave onset therefore, such monitors cannot be used as an input for impedance cardiography.
Current signal processing methods used in impedance cardiography and electrocardiography employ complex calculations and filtering techniques that necessarily impose significant hardware and software requirements. This restricts the ability to design a lightweight, portable, low cost monitoring device.

The present invention overcomes the aforementioned problems by offering an effective method and apparatus for simultaneous real-time electrophysiological and hemodynamic evaluation of cardiovascular fitness.

### BRIEF DESCRIPTION OF THE DRAWINGS

FIG. 1 illustrates basic concepts of thoracic impedance cardiography.

FIG. 2 shows a heart rate monitor with hemodynamic and electrophysiological assessment features.

FIG. 3 is a flow chart for calculation of heart rate, heart rate variability, stroke volume and cardiac output.

FIG. 4 illustrates characteristic points on ECG and ICG waveforms.

FIGS. 5a, 5b, 5c and 5d show detection and refining of point R.

FIG. 6 illustrates point and intervals of two successive RR intervals used in the calculation of noise level and point T.

FIGS. 7a and 7b illustrate saw-type and spike-type noise.

FIGS. 7c and 7d show ECG fragment before and after cubic spline smoothing.

FIGS. 8a, 8b and 8c illustrate detection of point Q.

FIGS. 9a, 9b and 9c illustrate detection of point S.

FIGS. 10a, 10b, 10c, 10d, 10e, 10f and 10g illustrate detection of points T and Tₑ.

FIG. 11 is a flow chart for obtaining (i) point C on an ICG and (ii)

$$\frac{ⅆ Z ( t )}{ⅆ {t}_{max}} .$$

FIG. 12 is a flow chart describing an iterative algorithm for filtering and smoothing ICG signals.

### DETAILED DESCRIPTION OF THE DRAWINGS

In the following description, numerous specific details are set forth to provide a thorough understanding of the present invention. However, it will be obvious to those skilled in the art that the present invention may be practiced without such specific details. For the most part, details concerning specific non-essential materials and the like have been omitted inasmuch as such details are not necessary to obtain a complete understanding of the present invention and are within the skills of persons of ordinary skill in the relevant art.

The fundamental aspect of the invention is a real-time simultaneous measurement of electrophysiological and hemodynamic parameters incorporated in a device, similar to the popular heart rate monitor, which can be used for comprehensive assessment of cardiovascular fitness.

These objectives are reached by using ECG signals, rather than ICG signals, for the measurement of key parameters needed for the calculation of electrophysiological and hemodynamic data, thus mitigating the influence of noise and artifacts on ICG signal.

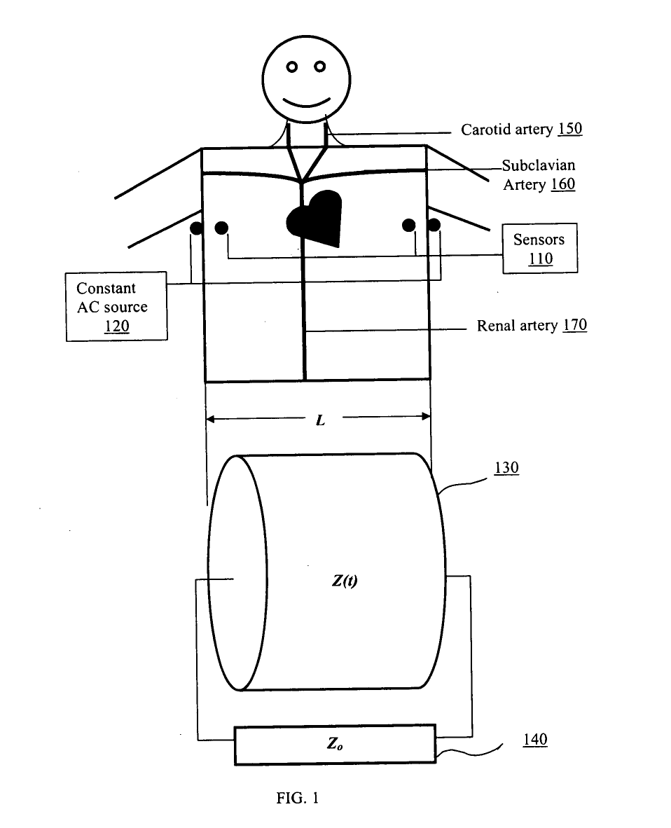

As shown in FIG. 1, AC current source electrodes 120 and sensing electrodes 110 are positioned across the torso substantially parallel to subclavian arteries 160. This configuration is in contrast to other thoracic bioimpedance set-ups whereby electrodes are placed along the torso in parallel to carotid 150 and renal arteries 170. According to equation (5), this positioning of electrodes provides lower resistivity Zₒ because, for the average person, the width of one's torso is less than the length of one's torso. therefore, the distance between electrodes (L) will be less

$$R = \frac{ρ L}{A} \qquad ( 5 )$$

Where:
R=resistivity of torso
L=the distance between sensing electrodes placed at opposite sides of the torso
A=cross section area of the torso (cm²)
ρ=resistivity of blood (ohms/cm)

Zₒ is considered equal to R because thoracic impedance technology is based on the assumption that the total thoracic impedance is totally resistive (i.e., the reactance component is equal zero). Consequently, Zₒ will be reduced providing better sensitivity to the time-varying component Z(t) of equation (2).

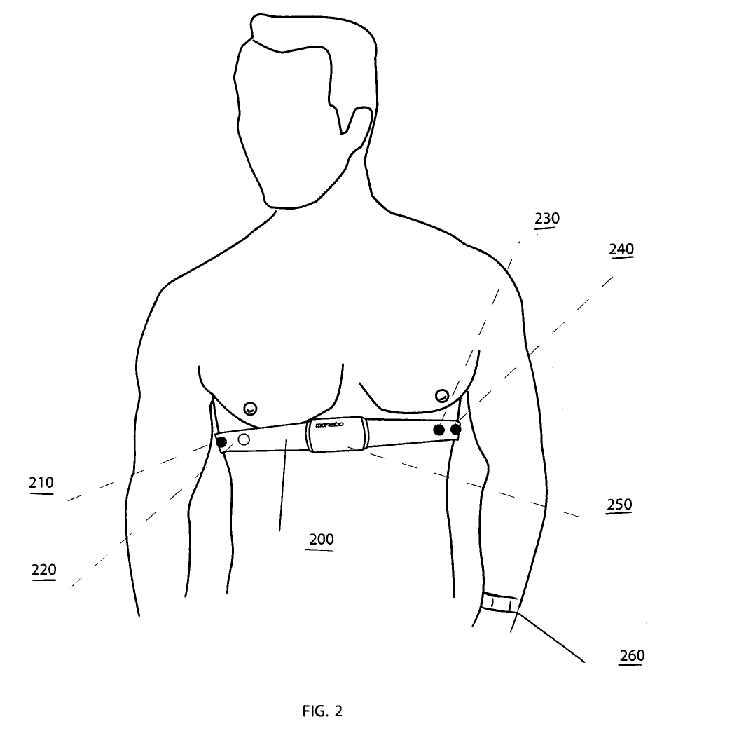

The heart rate monitor with hemodynamic and electrophysiological assessment capabilities is shown in FIG. 2 wherein a belt 200, with electrodes 210, 220, 230, and 240 and signal processing unit (“SPU”) 250 are shown. Electrodes 210 and 240 provide a path for constant alternating current (AC) application and electrodes 220 and 230 provide for voltage sensing. The electrodes 220 and 230 sense both the alternating current from electrodes 210 and 240 as well as the user's own ECG. Both of said signals are conveyed to the SPU 250, where they are filtered, amplified, digitized and transmitted to a wristwatch like device 260 or any other kind of processing unit for calculation and visualization of electrophysiological and hemodynamic parameters. Commonly used hardware filters, utilizing the difference of predefined frequencies for the ECG and ICG signals, work to separate the ECG and ICG signals. Electrodes 210, 220, 230, and 240 are placed in a flexible, stretchable belt that provides placement of the electrodes at specified locations regardless of the torso size.

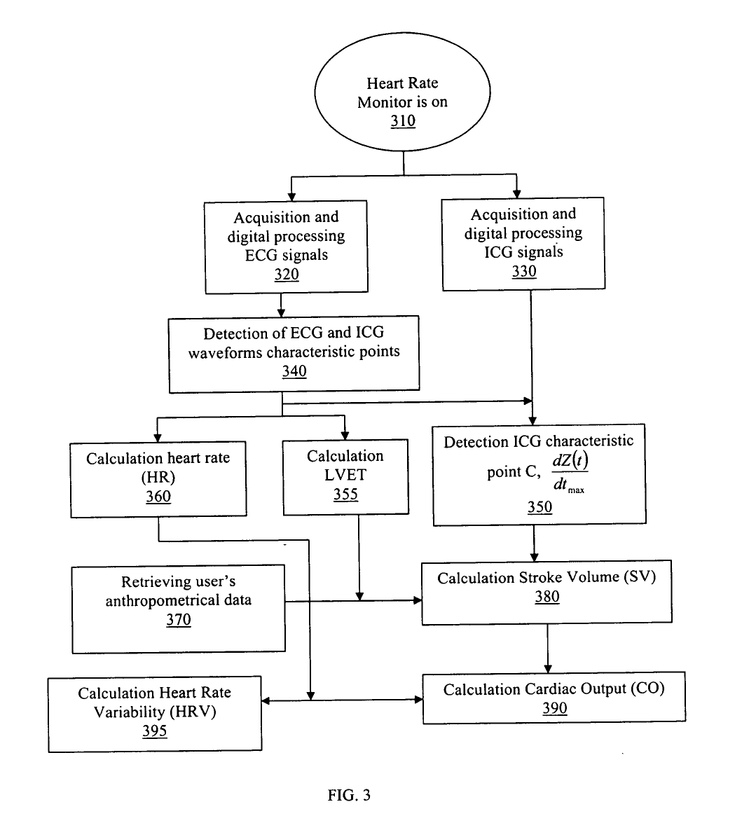

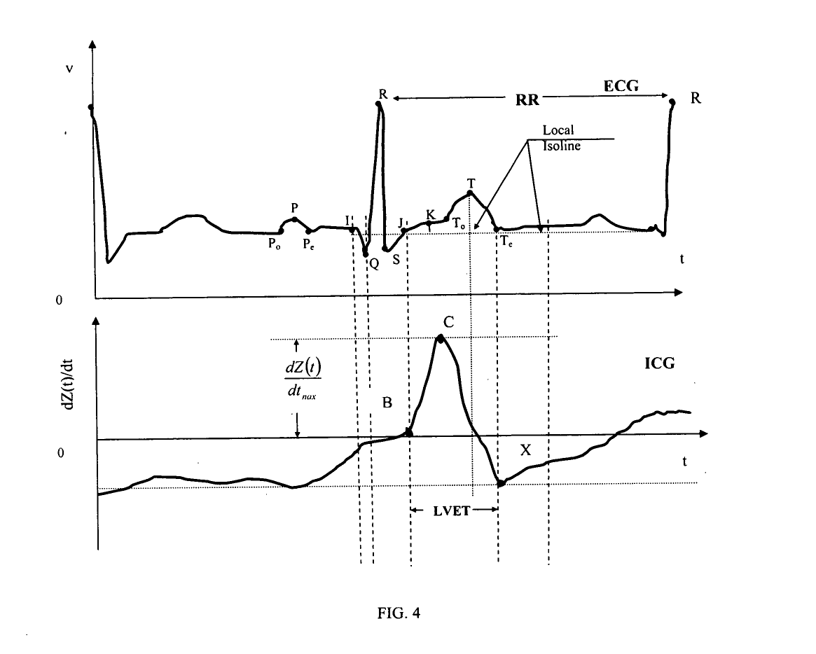

FIG. 3 illustrates how heart rate (HR), heart rate variability (HRV), stroke volume (SV), and cardiac output (CO) are calculated. After activating the heart rate monitor 310 and acquiring ECG 320 and ICG 330 signals, the characteristic points (a.k.a. fiduciary points) Q, R, S, J, T and Tₑ of ECG and fiduciary points B and X of ICG are ascertained in step 340 (FIGS. 3 and 4). Point C is ascertained on ICG in step 350. Point B corresponds to the time of the aortic valve opening while point X corresponds to the time of aortic valve closing. Point C corresponds to the maximum of

$$\frac{ⅆ Z ( t )}{ⅆ {t}_{max}} .$$

The time interval between point B and point X defines LVET. Because point B and X are hard to identify due to the low signal to noise ratio, the present method correlates point B with the time at which point J occurs. Furthermore, point X is correlated with the points in time when Tₑ occurs. Point C is much less vulnerable to noise and artifacts due to its distinct location. Consequently, point C may be measured directly on the ICG instead of being derived from points on the ECG.

The detection of characteristic points Q, R, S, J, T and Tₑ is illustrated in FIG. 4 to FIG. 10a-10e.

The detection of characteristic points starts from extraction of point R as the most distinctive point of ECG.

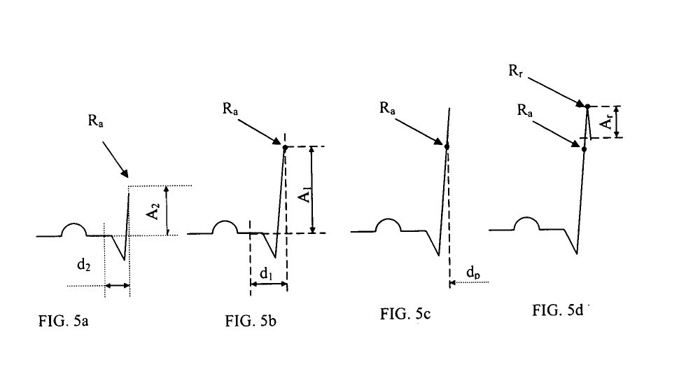

When progressing along axis t (FIG. 4) and comparing amplitude, Vᵢ, of a point at current time, tᵢ, and amplitudes, V₁ at time tᵢ−d₁ and V₂ at time tᵢ−d₂ (FIG. 5a-5d), the approximate location of point Rₐ is found, when the following is true:
(Vᵢ−V₁)>A₁ OR (Vᵢ−V₂)>A₂
Where:
Vᵢ=amplitude of the current point at time tᵢ;
V₁=amplitude at time tᵢ−d₁;
V₂=amplitude at time tᵢ−d₂;

A₁=0.25 mV and d₁=75 ms may be used for strongly expressed (high) R waves (5b).

A₂=0.15 mV and d₂=40 ms may be applied for weakly expressed (short) R waves (5a).

Theses values are commonly selected by those of skill in the art because the amplitude of point R normally increases 0.25 mV within a period of 75 ms for high R waves and it increases 0.15 mV within a period of 40 ms for short R waves. However, persons of ordinary skill in the art realize certain physiological conditions may require the alteration of values A₁, A₂, d₁ and d₂.

After a point Rₐ is found, the location Rₜ of point R is ascertained analyzing a time interval [tᵢ, t₁+dᵣ] (FIG. 5d). As soon as the amplitude of point R is decreases by more than Aᵣ, the location of point R is considered found and further analysis of interval [tᵢ, tᵢ+dᵣ] is stopped. The point Rᵣ is one step back from the point of decrease. The empiric values of dᵣ and Aᵣ are 200 ms and 0.05 mV respectively, because the maximum width of R wave is not more than 200 ms and, within this period, R wave descends by not less than 0.05 mV. However, persons of ordinary skill in the art may successfully use other values.

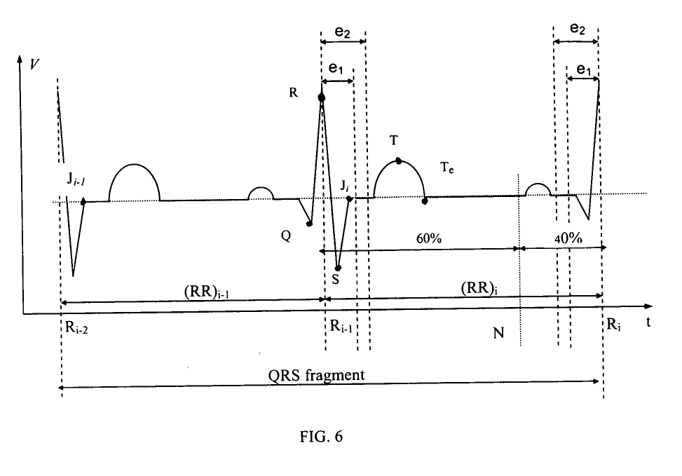

RR interval is the time between two successive R points (FIG. 6). QRS fragment is defined as two successive RR intervals (RR)ᵢ₋₁ and (RR)ᵢ (FIG. 6). Each current (RR)ᵢ interval is tested for the level of noise.

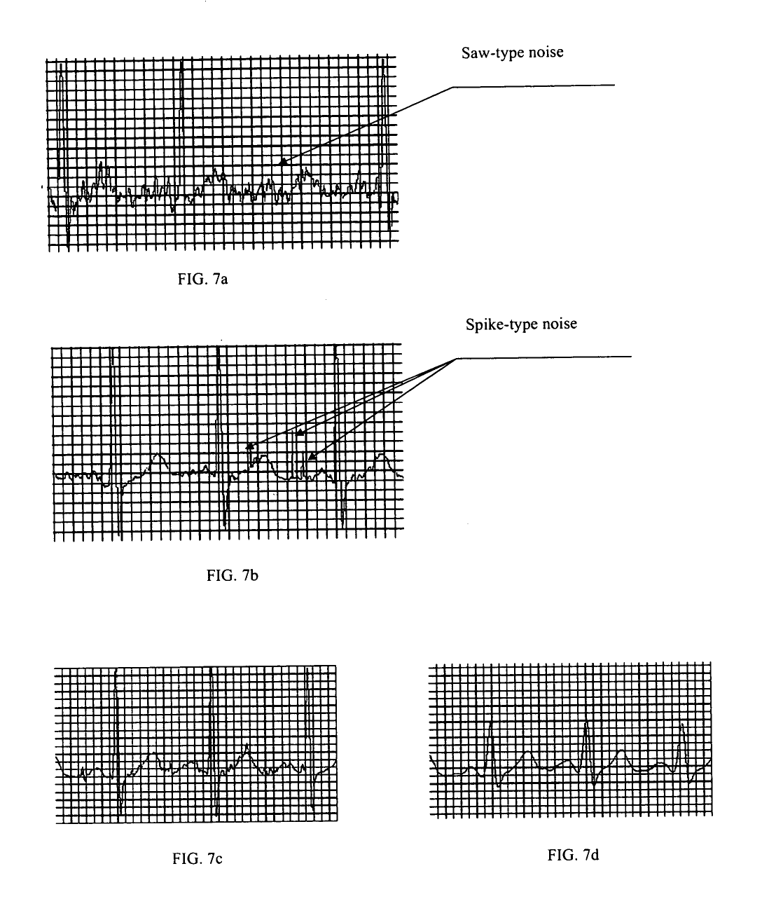

First the noise level, N₁, of saw-type noise (FIG. 7a) is calculated within time interval [Rᵢ₋₁+e₁, Rᵢ₋ₑ₁], (FIG. 6), where e₁ may typically be 75 ms.

Starting N₁=0, for each point j of interval [R₁₋₁+e₁, Rᵢ−e₁] and each m, if |Vⱼ−Vⱼ₋₁|>2^{m} AND |Vⱼ−Vⱼ₊₁|>2^{m}, then N₁=N₁+2^{m}, where m=3,2,1,0. At the next step the level N₂ of spike-type noise (FIG. 7b) is calculated within time interval [Rᵢ₋₁+e₁, Rᵢ−e₁], (FIG. 6b), where e₁ may typically be 115 ms.

Starting N₂=0, for each point j of interval [Rᵢ₋₁+e₁, Rᵢ−e₁] and each m, if |Vⱼ−Vⱼ₋₁>m AND |Vⱼ−Vⱼ₊₁ |>m, then N₁=N₁+m, where m=30, 20.

The total noise level of current interval (RR)ᵢ, Nᵢ=N₁+N₂. If the noise level Nᵢ>Nₗᵢₘᵢₜ where Nₗᵢₘᵢₜ may be 20, then current interval (RR)ᵢ is considered unreliable and excluded from further calculations.

The values e₁=75 ms and e₂=115 ms are empirically derived and commonly selected by those of skill in the art because indentations 75 ms and 115 ms from R point exclude Q, S and T waves from mistakenly considering these points as saw-type noise as well as point R as a spike-type noise. However, persons of ordinary skill in the art may successfully use other values.

Nₗᵢₘᵢₜ=20 provides a sufficient noise filtering for disclosed application however, persons of ordinary skill in the art may successfully use other values.

After noise filtering of RR interval, RR interval is smoothed using cubic spline interpolation algorithm included in Matlab Version 3.2 spline toolbox. However, persons of ordinary skill in the art may successfully use other smoothing techniques. FIG. 7c illustrates ECG fragment before smoothing, while FIG. 7d shows ECG fragment after cubic spline smoothing was applied.

QRS fragment (FIG. 6) is defined as two successive reliable RR intervals, (RR)ᵢ₋₁ and (RR)ᵢ.

Point Q, S, J, T, and Tₑ are ascertained within QRS fragment.

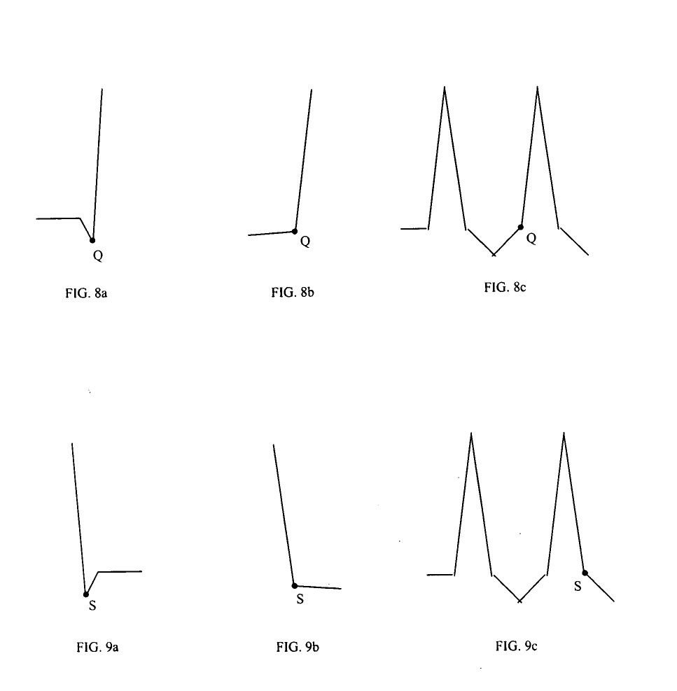

Point Q is calculated from the graphs in FIGS. 8a-8c in the following manner:

Referring to FIGS. 8a and recalling that R has already been located as shown above, point Q may be obtained as follows:

A time interval to be analyzed is defined as [t_{DQ}, t_{R}] where, typically, t_{DQ}=t_{R}−75 ms. t_{R} corresponds with point R as was derived above. A 75 ms period is commonly selected by those of skill in the art because the onset of Q wave is normally within 0 to 75 ms of R. However, persons of ordinary skill in the art may select other value, which may be successfully applied.

When sampling this period, starting at t_{R} and progressing towards t_{DQ}, Q is found when the following is true:
A_{i-1> and (}A_{R}−Aᵢ)>A_{RQ}
Where:
A denotes amplitude
A_{RQ}=0.1 mV
A_{R}=the amplitude at point R

A_{RQ} =0.1 mV is commonly selected by those of skill in the art because a typical R peak is at least 0.1 mV “above” the Q. However, persons of ordinary skill in the art may select other value, which may be successfully applied. This approach is valid for normal Q waves as shown on FIG. 8a.

Next, if the above conditions are not met and Q is not ascertained, the following conditions are evaluated to determine Q (See FIG. 8b):

A time interval to be analyzed is defined as [t_{DQ}, t_{R}] where, again, typically, t_{DQ}=t_{R}−75 ms. A 75 ms period is commonly selected by those of skill in the art because the onset of Q wave is normally within 0 to 75 ms of R. However, persons of ordinary skill in the art may select other value, which may be successfully applied.

When sampling this period, starting at t_{R} and progressing towards t_{DQ}, Q is found when the following is true:
(A−Aᵢ₋₃)>A_{d} and (A_{R}−Aᵢ)>A_{RQ}
Where:
A denotes amplitude
A_{d}=0.025 mV
A_{RQ}=0.1 mV

A_{RQ}=0.1 mV is commonly selected by those of skill in the art because a typical R peak is at least 0.1 mV “above” the Q. However, persons of ordinary skill in the art may select other value, which may be successfully applied.

A_{d}=0.025 mV is commonly selected by those of skill in the art because this amplitude difference is typical for abnormal Q wave shown on FIG. 8b. However, persons of ordinary skill in the art may select other value, which may be successfully applied.

Next, if the above conditions are not met and Q is not ascertained, the following conditions are evaluated to determine Q (See FIG. 8c):

A time interval to be analyzed is defined as [t_{DQ}, t_{R}] where, again, typically, t_{DQ}=t_{R}−75 ms. A 75 ms period is commonly selected by those of skill in the art because the onset of Q wave is normally within 0 to 75 ms of R. However, persons of ordinary skill in the art may select other value, which may be successfully applied.

When sampling this period, starting at t_{R} and progressing towards t_{DQ}, Q is found when the following is true:

$$\frac{{A}_{i} - {A}_{i - 3}}{{A}_{i + 3} - {A}_{i}} \geq {Q}_{r} and ( {A}_{R} - {A}_{i} ) > {A}_{RQ}$$

Where:
A denotes amplitude
A_{RQ}=0.1 mV
Qᵣ=0.45

A_{RQ}=0.1 mV is commonly selected by those of skill in the art because a typical R peak is at least 0.1 mV “above” the Q. However, persons of ordinary skill in the art may select other value, which may be successfully applied.

Qᵣ=0.45 is commonly selected by those of skill in the art because it's a typical ratio for abnormal Q wave related to group of premature beats (FIG. 8c). However, persons of ordinary skill in the art may select other value, which may be successfully applied.

Referring to FIG. 9a and recalling that R has already been located as shown above, point S may be obtained as follows:

A time interval to be analyzed is defined as [t_{R}, t_{DS}] where, typically, t_{DS}=t_{R}+75 ms. A 75 ms period is commonly selected by those of skill in the art because the onset of S wave is normally within 0 to 75 ms of R. However, persons of ordinary skill in the art may select other value, which may be successfully applied.

When sampling this period, starting at t_{R} and progressing towards t_{DS}, S is found when the following is true:
A₁₊₁>Aᵢ and (A_{R}−Aᵢ)>A_{RS}
Where:
A denotes amplitude
A_{RS}=0.1 mV

A_{RS}=0.1 mV is commonly selected by those of skill in the art because a typical R peak is at least 0.1 mV “above” the S. However, persons of ordinary skill in the art may select other value, which may be successfully applied. This approach is valid for normal S waves as shown on FIG. 9a.

Next, if the above conditions are not met and S is not ascertained, the following conditions are evaluated to determine S (See FIG. 9b):

A time interval to be analyzed is defined as [t_{R}, t_{DS}] where, again, typically, t_{DS}=t_{R}+75 ms. A 75 ms period is commonly selected by those of skill in the art because the onset of S wave is normally within 0 to 75 ms of R. However, persons of ordinary skill in the art may select other value, which may be successfully applied.

When sampling this period, starting at t_{R} and progressing towards t_{DS}, S is found when the following is true:
(Aᵢ−Aᵢ₊₃)<A_{d} and (A_{R}−Aᵢ)>A_{RS}
Where:
A denotes amplitude
A_{d}=0.025 mV
A_{RS}=0.1 mV

A_{RS}=0.1 mV is commonly selected by those of skill in the art because a typical R peak is at least 0.1 mV “above” the S. However, persons of ordinary skill in the art may select other value, which may be successfully applied.

A_{d}=0.025 mV is commonly selected by those of skill in the art because a this amplitude difference is typical for abnormal S wave shown on FIG. 9b. However, persons of ordinary skill in the art may select other value, which may be successfully applied.

Next, if the above conditions are not met and S is not ascertained, the following conditions are evaluated to determine S (See FIG. 9c):

A time interval to be analyzed is defined as [t_{R}, t_{DS}] where, again, typically, t_{DS}=t_{R}+75 ms. A 75 ms period is commonly selected by those of skill in the art because the onset of S wave is normally within 0 to 75 ms of R. However, persons of ordinary skill in the art may select other value, which may be successfully applied.

When sampling this period, starting at t_{R} and progressing towards t_{DS}, S is found when the following is true:

$$\frac{{A}_{i} - {A}_{i + 3}}{{A}_{i - 3} - {A}_{i}} \geq {S}_{r} and ( {A}_{R} - {A}_{i} ) > {A}_{RS}$$

Where:
A denotes amplitude
A_{RS}=0.1 mV
Sᵣ=0.3

A_{RS}=0.1 mV is commonly selected by those of skill in the art because a typical R peak is at least 0.1 mV “above” the S. However, persons of ordinary skill in the art may select other value, which may be successfully applied.

Sᵣ=0.3 is commonly selected by those of skill in the art because it's a typical ratio for abnormal S wave related to group of premature beats (FIG. 9c). However, persons of ordinary skill in the art may select other value, which may be successfully applied.

Recalling that point S has already been located as shown above, point J (FIG. 4) may be obtained as follows:

A time interval to be analyzed is defined as [t_{S}, t_{DJ}] where, typically, t_{DJ}=t_{S}+75 ms. A 75 ms period is commonly selected by those of skill in the art because the point J is normally within 0 to 75 ms of S. However, persons of ordinary skill in the art may select other value, which may be successfully applied.

When sampling this period, starting at t_{R} and progressing towards t_{DJ}, J is found when the following is true:
(Aᵢ₊₃−Aᵢ)<A_{d}
Where:
A denotes amplitude
A_{d}=0.025 mV

If point J is not ascertained, point J may be considered to have time coordinate equal t_{Q}+50 ms. t_{Q}+50 ms is selected because the point J normally coincides with the onset of Pre-ejection Period, which normally starts within 50 ms after point Q.

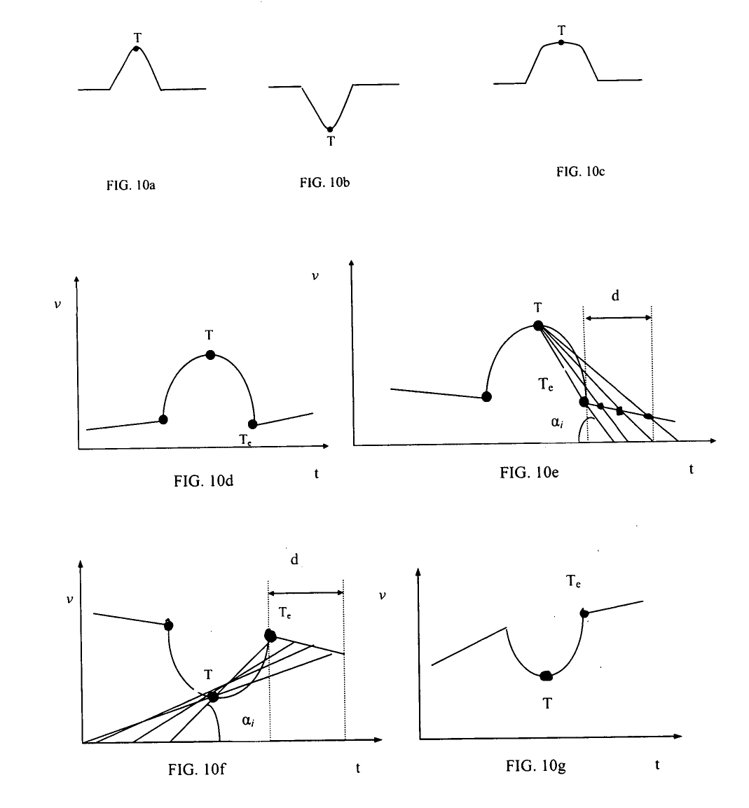

Recalling that point J (FIG. 4) has been located and two successive RR intervals, (RR)ᵢ₋₁ and (RR)ᵢ (FIG. 6) have been identified as shown above, point T (FIG. 4, FIGS. 10a-10e) may be obtained as follows:

A time interval to be analyzed is defined as [t_{J}, t_{N}], where t_{N}=60%*[Rᵢ₋₁, Rᵢ] (FIG. 6). 60% is commonly selected by those of skill in the art, because the point T is normally located within interval [t_{J}, t_{N}]. However, persons of ordinary skill in the art may select other value, which may be successfully applied.

When sampling this period, starting at t_{J} and progressing towards t_{N}, point T is found if the distance from moving point (tᵢ, Aᵢ) to straight line (Jᵢ, Jᵢ₋₁), which exists between point Jᵢ of interval [Rᵢ₋₂, Rᵢ] and point Jᵢ₋₁ of interval [Rᵢ₋₁, Rᵢ₋₁] (FIG. 6), is more than T_{Amax}. Where T_{Amax} is a maximum point and=2 mm. Those of skill of the art commonly select this value, however, persons of ordinary skill in the art may select other value, which may be successfully applied. This approach is valid for normal T wave as it is shown on FIG. 10a.

If T wave is inverted (FIG. 10b), then, when sampling this period, starting at t_{J} and progressing towards t_{N}, point T is found if the distance from moving point (tᵢ, Aᵢ) to straight line, (Jᵢ, Jᵢ₋₁), which exists between point Jᵢ of interval [Rᵢ₋₂, Rᵢ] and point Jᵢ₋₁ of interval [Rᵢ₋₁, Rᵢ₋₁] (FIG. 6), is more than T_{Amin}. Where T_{Amin} is a minimum point and=8 mm. This value is commonly selected by those of skill of the art, however, persons of ordinary skill in the art may select other value, which may be successfully applied. This approach is valid for a normal T wave as it is shown on FIG. 10b.

For flat T waves (FIG. 10c), sampling interval [tⱼ, t_{N}] as defined above and progressing toward t_{N}, point T is found when the following is true:
(Aᵢ−Aᵢ₊₅)>A_{d}
Where:
A denotes amplitude
A_{d}=0.025 mV

A_{d}=0.025 mV is commonly selected by those of skill in the art because experimental data well correlated with this value. However, persons of ordinary skill in the art may select other value, which may be successfully applied.

If point T is not identified, the point is considered undetectable. Time coordinates of point T and point Tₑ are then considered equal to t_{N}.

When point T has been located, then point Tₑ (FIGS. 10d, 10e) is ascertained as follows:

A time interval to be analyzed starting from t_{T} and progressing to direction of time gain.

Point Tₑ is found when one of the following is true:
Aᵢ>Aᵢ₋₁, if T wave has shape as shown on FIG. 10d
or
Aᵢ<Aᵢ₋₁, if T wave has shape as shown on FIG. 10f
or
angle αᵢ, between straight line (T, Tᵢ) and axis t, becomes less than αᵢ₋₁ and this condition remains for the period of time not less then d=40 ms for T waves shaped as shown on FIG. 10e and FIG. 10g.

d=40 ms is commonly selected by those of skill in the art because experimental data well correlates with this value. However, persons of ordinary skill in the art may select other value, which may be successfully applied.

Other methods such as spectral analysis, signal averaging, wavelet transform, and Fourier transform may be successfully used for detection of characteristic points of ECG.

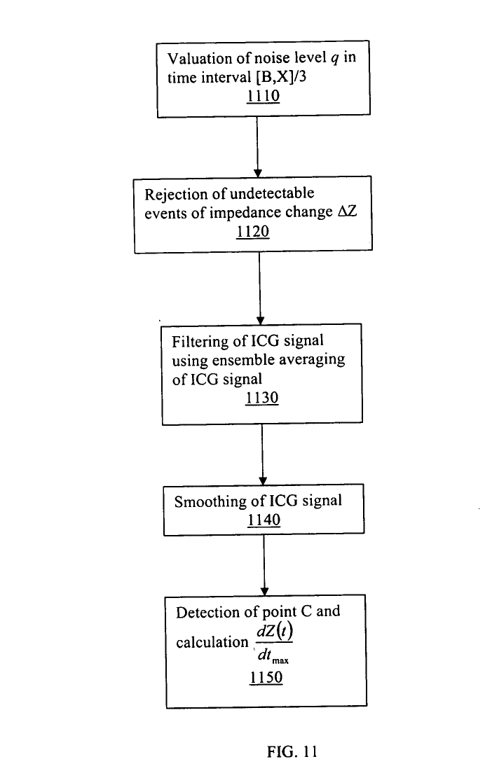

FIG. 11 illustrates detection of point C, which defines parameter

$$\frac{ⅆ Z ( t )}{ⅆ {t}_{max}}$$

of equations (10). First, noise level q is identified 1110. The noise level q is defined as the average change of ICG signals between two adjacent points within n time intervals [B,X]/3 using formula (6):

$$q = \frac{{\sum }_{1}^{n - 1} \vert {Y}_{j} - {Y}_{j + 1} \vert}{2 n} \qquad ( 6 )$$

Where:
q=noise level of ICG signal;
j=1 to n−1, where n is total number of points (samples) in interval [B,X]/3;
Yⱼ=j^{th} point (sample) of ICG signal.
n=the number of points within time interval [B,X1/3;
   n=F*[B,X]/3

Where F is the sampling frequency (number of samples per second) of digitization performed in the processing unit 260. Typical sample frequencies are from 200 Hz to 1,000 Hz.

Next, to further ensure a signal with a viable point C has been obtained, impedance change, ΔZ 1120 is deemed undetectable if:

$$\frac{Δ Z}{2 {Z}_{o} + Δ Z} < \frac{q}{2 {Z}_{o}} \qquad ( 7 )$$

Where:
ΔZ=impedance change;
Zₒ=base impedance;
q=noise level
   ΔZ=k*Zₒ

In the present embodiment, k=0.01, although other values may be successfully used. k is typically in a range of 0.005 to 0.02.

In subsequent steps, acceptable ICG signals are qualified using ensemble averaging 1130 and then subjected to smoothing processes 1140. Point C is then calculated as the peak height

$$\frac{ⅆ Z ( t )}{ⅆ t}$$

on the ICG curve 1150 within time interval [B, X], which is equal to interval [J, Tₑ]. The number of consecutive [B,X]ᵢ intervals n included in the ensemble averaging process 1130 depends on a predefined threshold signal-to-noise ratio (SNR), N, and on a permissible number, M, of consecutive [B,X] intervals used for ensemble averaging. In one embodiment, N=1,000 and M=10, although other values may be successfully used.

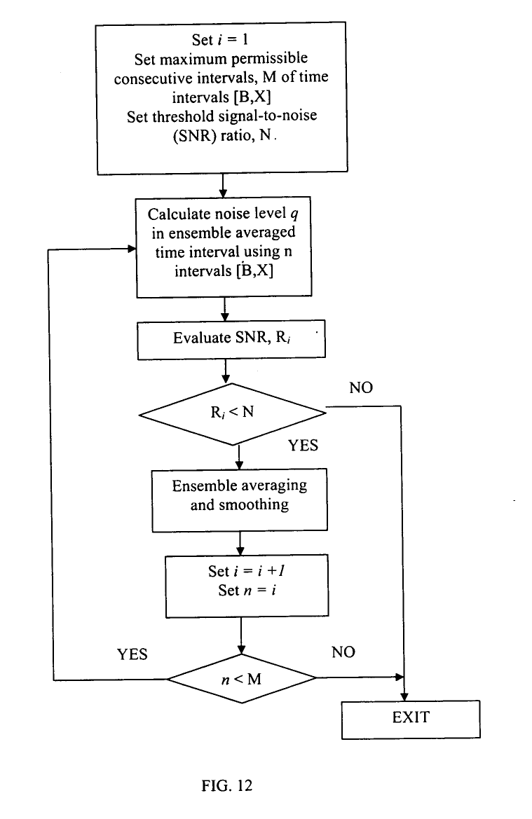

FIG. 12 illustrates iterative algorithm of filtering and smoothing of ICG signals.

Each point Sⱼ is calculated as the averaged value of correspondent points of n consecutive [B,X]ᵢ intervals (8):

$${S}_{j} = \frac{{\sum }_{1}^{n} {Y}_{{j}_{i}}}{n} \qquad ( 8 )$$

The expected improvement in signal-to-noise ratio after ensemble averaging process is expressed by \sqrt{n}.

The smoothing of the ICG signal 1140 in interval [B,X] is performed using triangular 5-points smooth method. The signal value, Sⱼ, is calculated by equation (9):

$${S}_{j} = \frac{{Y}_{j - 2} + 2 {Y}_{j - 1} + 3 {Y}_{j} + 2 {Y}_{j + 1} + {Y}_{j + 2}}{9} \qquad ( 9 )$$

for j=3 to n−2, where Sⱼ is the j^{th} point in the smoothed signal, Yⱼ is the j^{th} point in the original signal, and is the total number of samples (points) in the interval [B,X]. The process increases the signal-to-noise ratio by \sqrt{5}.

The total improvement of signal-to-noise ratio after ensemble averaging and smoothing is \sqrt{5}*n.

Returning to FIG. 3, and aside from calculating point C, Left Ventricular Ejection Time (LVET) 355 must be calculated. Instead of using point B and X of ICG, the method uses point J, the point of junction between the S wave and T wave, and point Tₑ, which is at the end of the T wave of the ECG. Point J corresponds to aortic valve opening and point Tₑ corresponds aortic valve closing. LVET is equal to the time interval between point J and point Tₑ of the ECG waveform. Choosing points J and Tₑ provides more reliable and reproducible results, because the exact time domain location of points J and Tₑ are much easier to identify than the transient location of points B and X of an ICG. Also, calculation of LVET as the interval between points J and Tₑ, automatically corrects LVET which may be significantly affected by increased or decreased heart rate.

Heart rate is calculated 360 (FIG. 3) as a moving average of n consecutive RR intervals, where n is derived from iterative filtering and smoothing process 1130 and 1140 described in FIG. 12.

Anthropometrical data 370 comprise of torso height and torso perimeter.

Calculation of stroke volume 380 may employ any of the equations (3) and (4) or their modifications. In the present invention, stroke volume is calculated using Gundarov modified equation (10).

$$SV = 0.9 ρ κ \frac{{H}^{2} L}{{Z}_{o}^{2}} LVET \frac{ⅆ Z ( t )}{ⅆ {t}_{max}} \qquad ( 10 )$$

Where:
L=distance between sensing electrodes (cm),
LVET=left ventricular ejection time,
Zₒ=base impedance (ohms),

$$\frac{ⅆ Z ( t )}{ⅆ {t}_{max}} = magnitude of the largest negative derivative of the impedance change ,$$
Z(t) occurring during systole (ohms/s),
ρ=resistivity of blood (ohms/cm),
H=height of the user's torso,
κ=torso correction factor.

Torso correction factors (κ) are shown in the table (2):

TABLE 2

| Torso size in cm | k |
|---|---|
| 80 | 0.0036 |
| 82 | 0.0035 |
| 84 | 0.0034 |
| 86 | 0.0033 |
| 88 | 0.0032 |
| 90 | 0.0032 |
| 92 | 0.0031 |
| 94 | 0.0030 |
| 96 | 0.0029 |
| 98 | 0.0028 |
| 100 | 0.0027 |
| 102 | 0.0027 |
| 104 | 0.0026 |
| 106 | 0.0025 |
| 108 | 0.0025 |
| 110 | 0.0024 |

Once stroke volume 380 has been calculated, cardiac output 390 is calculated using equation (1).
CO=SV*HR
Where:
CO=cardiac output
SV=stroke volume
HR=heart rate

Although the present invention and its advantages have been described in detail, it should be understood that various changes, substitutions and alterations can be made herein without departing from the spirit and scope of the invention.

1. A method of assessing the cardiovascular fitness of a user, comprising:
a) Placing a plurality of electrodes on said user's torso;
b) Applying a current to said torso using at least one of said electrodes;
c) Acquiring an ECG using at least one of said electrodes;
d) Acquiring an ICG using at least one of said electrodes;
e) Identifying one or more characteristic points on said ECG;
f) Identifying one or more characteristic points on said ICG;
g) Measuring parameters using at least one of said one or more characteristic points on said ECG; and
h) Measuring parameters using at least one of said one or more characteristic points on said ICG.

2. The method of claim 1, wherein said plurality of electrodes comprises two sensing electrodes positioned substantially parallel to the subclavian artery.

3. The method of claim 2, wherein said sensing electrodes are placed in a stretchable strap.

4. The method of claim 2, wherein said sensing electrodes acquire an ECG and an ICG.

5. The method of claim 1, wherein said plurality of electrodes comprise two electrodes for providing alternating current.

6. The method of claim 5, wherein said two electrodes for providing alternating current are placed in a stretchable strap.

7. The method of claim 1, wherein said step of identifying one or more characteristic points on said ECG entails identifying at least one of the following points: R, Q, S, J, T and Tₑ.

8. The method of claim 1, wherein said step of identifying one or more characteristic points on said ICG entails identifying at least one of the following points: B, C and X.

9. The method of claim 8, wherein said point B is substantially equal to ECG point J.

10. The method of claim 9, wherein said point X is substantially equal to ECG point Tₑ.

11. The method of claim 10, wherein points J and Tₑ are used for LVET calculation.

12. The method of claim 8, wherein said point C is approximately the peak point of said ICG within time interval [J, Tₑ] and further wherein said point C defines
$$\frac{ⅆ Z ( t )}{ⅆ {t}_{max}} .$$

13. The method of claim 12, wherein said detection of said point C comprises calculation of noise level, rejection of undetectable events of impedance change, ensemble averaging process and 5-points triangular smoothing process.

14. The method of claim 13, wherein said valuation of noise level is performed using the formula:
$$q = \frac{{\sum }_{1}^{n - 1} \vert {Y}_{j} - {Y}_{j + 1} \vert}{2 n}$$
Where:
q=noise of ICG signal;
j=1 to n−1, where n is total number of samples in interval [B,X]/3;
Yⱼ=j^{th} point of ICG signal.

15. The method of claim 13, wherein said event of impedance change is considered undetectable if:
$$\frac{Δ Z}{2 {Z}_{o} + ΔZ} < \frac{q}{2 {Z}_{o}}$$
Where:
ΔZ=impedance change;
Zₒ=base impedance;
q =noise level
ΔZ=k*Zₒ

16. The method of claim 13, wherein said ensemble averaging process is performed using n number of consecutive [B, X] intervals, where n is derived from iterative filtering and smoothing algorithm.

17. The method of claim 16, wherein said iterative filtering and smoothing algorithm uses the threshold signal-to-noise ratio, N, and the threshold number, M, of permissible consecutive intervals, n.

18. The method of claim 1, wherein said step of measuring parameters using at least one of said one or more characteristic points on said ECG; and further wherein said step of measuring parameters using at least one of said one or more characteristic points on said ICG yields at least one of the following: heart rate, heart rate variability, stroke volume and cardiac output.

19. The method of claim 19, wherein said stroke volume is calculated using:
$$SV = 0.9 ρκ \frac{ {H}^{2} L}{{Z}_{o}^{2}} LVET \frac{ⅆ Z ( t )}{ⅆ {t}_{max}}$$
Where:
L=distance between sensing electrodes in cm,
LVET=left ventricular ejection time,
Zₒ=base impedance in ohms,
$$\frac{ⅆ Z ( t )}{ⅆ {t}_{max}} = magnitude of the largest$$
$$negative derivative of the impedance change ,$$
Z(t) occurring during systole in ohms/s,
ρ=resistivity of blood in ohms/cm, and
κ=torso correction factor.
